(19) United States
(12) Patent Application Publication
(10) Pub. No.: US 2015/0133819 A1
(43) Pub. Date: May 14, 2015

(54) SYSTEM AND METHOD OF PERFORMING AUSCULTATION USING MOTOR VEHICLE SEATS

(71) Applicants: Hyundai America Technical Center, Inc., Superior Township, MI (US); Kia Motors Corporation, Seoul (KR); Hyundai Motor Company, Seoul (KR)

(72) Inventor: Chadd Price, Livonia, MI (US)

(73) Assignees: Hyundai America Technical Center, Inc., Superior Township, MI (US); Kia Motors Corporation, Seoul (KR); Hyundai Motor Company, Seoul (KR)

(21) Appl. No.: 14/075,205

(22) Filed: Nov. 8, 2013

(51) Int. Cl. A61B 7/04

(52) U.S. Cl. CPC A61B 7/04
USPC 600/586

(57) ABSTRACT

According to the disclosed embodiments, auscultation is performed using motor vehicle seats. The embodiments include a vehicle seat including a seat support member that includes at least one mounting rod extending in a longitudinal direction, and an auscultation device. The auscultation device includes at least one microphone that is movably coupled to the at least one mounting rod and configured to perform a screening of a passenger in the vehicle seat by capturing a bodily sound of the passenger. A controller is then configured to initiate the screening of the passenger and to diagnose the passenger based on results of the screening.

### TECHNICAL FIELD

The present disclosure relates generally to motor vehicles, and, more particularly, to performing auscultation using motor vehicle seats.

### BACKGROUND

For much of society, maintaining a healthy lifestyle is an essential consideration. Significant portions of the population are aging and experiencing declining health, or suffer from a wide range of illnesses including the cold, flu, and so forth. Many of these conditions may be diagnosed and any potential harm mitigated through regular check-ups with a medical professional. In particular, the conditions may be accurately examined through auscultation methods, i.e., listening to internal sounds of the body produced by, for example, the respiratory system (e.g., heart and lungs) and the gastrointestinal system (e.g., digestive system/bowels). Despite the importance of maintaining a healthy lifestyle, however, many people are simply too busy to receive regular medical check-ups. It is often the case that healthcare-related considerations are eschewed in view of one's daily obligations relating to, e.g., work, family, etc. Therefore, there is currently a need to provide a manner in which people may receive regular, effective medical diagnosis check-ups, while minimizing interference with other non-health-related obligations.

### SUMMARY

According to the disclosed embodiments, auscultation is performed using motor vehicle seats. Many people drive their vehicles daily, thus providing for an ideal time and location to perform routine health screenings using auscultation methods to diagnose issues relating to, e.g., the heart, lungs, bowels, etc. The embodiments include a vehicle seat including a seat support member that includes at least one mounting rod extending in a longitudinal direction, and an auscultation device. The auscultation device includes at least one microphone that is movably coupled to the at least one mounting rod and configured to perform a screening of a passenger in the vehicle seat by capturing a bodily sound of the passenger. A controller is then configured to initiate the screening of the passenger and to diagnose the passenger based on results of the screening. By doing so, the vehicle can detect and assess the health of the driver.

### BRIEF DESCRIPTION OF THE DRAWINGS

The foregoing and other objects, features, aspects and advantages of the embodiments disclosed herein will become more apparent from the following detailed description when taken in conjunction with the following accompanying drawings.

FIG. 1 illustrates an example schematic diagram of a controller operable to control a system and method of performing auscultation using motor vehicle seats.

FIGS. 2A and 2B illustrate an example simplified configuration of a vehicle seat comprising an auscultation device.

FIGS. 3A and 3B illustrate a side view of an example simplified configuration of the auscultation device.

FIG. 4 illustrates an example simplified configuration of a positioning assistance device.

FIG. 5 illustrates an example simplified procedure for calibrating the auscultation device and initiating a screening of a passenger.

FIG. 6 illustrates an example simplified procedure for performing the screening of the passenger and determining a diagnosis of the passenger based on results of the screening.

It should be understood that the above-referenced drawings are not necessarily to scale, presenting a somewhat simplified representation of various preferred features illustrative of the basic principles of the disclosure. The specific design features of the present disclosure, including, for example, specific dimensions, orientations, locations, and shapes, will be determined in part by the particular intended application and use environment.

### DETAILED DESCRIPTION OF THE EMBODIMENTS

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). Along the same lines, the term “passenger” or “vehicle passenger” refers to any person located inside a vehicle, including, for example, a driver, a rider in the front passenger seat or backseat, etc.

Additionally, it is understood that a number of the below methods are executed by at least one controller. The term “controller” refers to a hardware device that includes a memory and a processor. The memory is configured to store program instructions and the processor is specifically configured to execute said program instructions to perform one or more processes which are described further below.

Furthermore, the controller of the present disclosure may be embodied as non-transitory computer readable media on a computer readable medium containing executable program instructions executed by a processor, controller or the like. Examples of the computer readable mediums include, but are not limited to, ROM, RAM, compact disc (CD)-ROMs, magnetic tapes, floppy disks, flash drives, smart cards and optical data storage devices. The computer readable recording medium can also be distributed in network coupled computer systems so that the computer readable media is stored and executed in a distributed fashion, e.g., by a telematics server or a Controller Area Network (CAN).

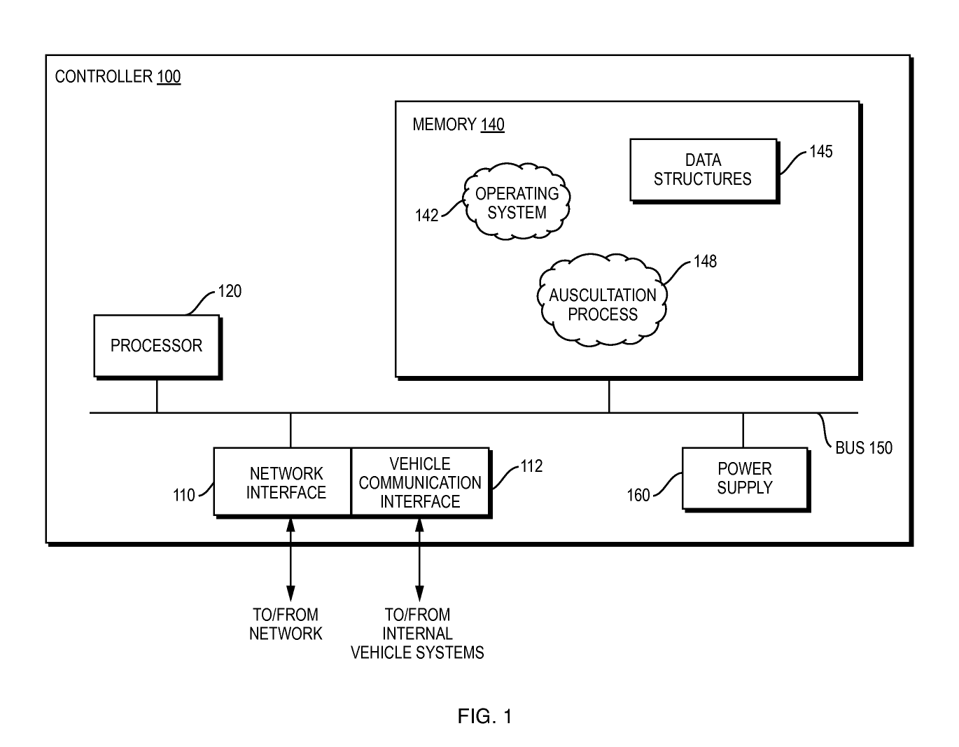

FIG. 1 illustrates an exemplary schematic diagram of a controller operable to control a system and method of performing auscultation using motor vehicle seats. More specifically, the controller 100 is configured to at least initiate a screening of a passenger in a vehicle seat by an auscultation device and to determine a diagnosis of the passenger based on results of the screening, as described in further detail below. As shown in FIG. 1, the controller 100 may comprise a network interface 110, a vehicle communication interface 112, a processor 120, a memory 140, and a power supply 160 (e.g., battery), all of which may be interconnected by a system bus 150. Note that the power supply 160 is depicted inside the controller 100 merely for illustration purposes and may not actually reside “inside” the controller.

The network interface 110, e.g., transceivers, network adaptors, wireless cards, etc. contain the mechanical, electrical, and/or signaling circuitry for communicating data over links, e.g., wired/physical, wireless, or otherwise, coupled to a remote network, including, for example, a data repository, a hospital network, the cloud, etc. The network interface 110 may be configured to transmit and/or receive data using a variety of different communication protocols, as will be understood by those skilled in the art. One noted means communication that may be preferably applied to an exemplary embodiment is a CAN system, which are typically integrated into a vehicle's network. Thus, the exemplary embodiment may be configured to interface accordingly. The controller 100 may have multiple different types of network interfaces 110, e.g., wireless and wired/physical connections, whereby the view herein is merely for illustration.

Similar to the network interface 110, the vehicle communication interface 112 contains the mechanical, electrical, and/or signaling circuitry for communicating data over links, e.g., wired/physical, wireless, or otherwise, coupled to any internal vehicle systems. The internal vehicle systems represent systems within the vehicle communicable with the controller 112, such as, for example, an in-vehicle auscultation device, a passenger notification system configured to, for example, provide audible/visual notifications to a passenger (e.g., diagnoses, calibration information, warnings/recommendations, etc.), and so forth. The internal vehicle systems referenced above are described in further detail below. The vehicle communication interface 112 may be configured to transmit and/or receive data using a variety of different communication protocols, as will be understood by those skilled in the art.

The memory 140 comprises a plurality of storage locations that are addressable by the processor 120, the network interface 110, and the vehicle communication interface 112 for storing data, including programs, data structures, and the like, associated with the embodiments described herein. The processor 120 may comprise necessary elements or logic adapted to execute any stored programs and manipulate the data structures 145. An operating system 142, portions of which are typically resident in memory 140 and executed by the processor 120, functionally organizes the device by, inter alia, invoking operations in support of processes and/or services executing on the device. These processes and/or services may comprise an illustrative “auscultation process” 148, as described in greater detail below.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules configured to operate in accordance with the techniques herein (e.g., according to the functionality of a similar process). Further, while the processes have been shown separately, those skilled in the art will appreciate that processes may be routines or modules within other processes.

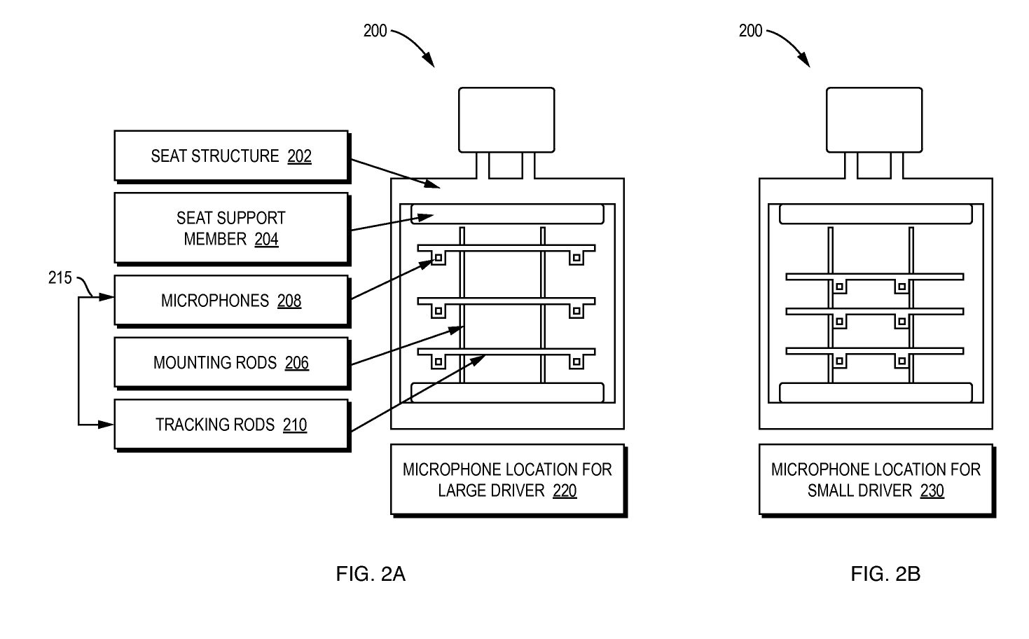

FIGS. 2A and 2B illustrate an exemplary simplified configuration of a vehicle seat comprising an auscultation device. As shown in FIGS. 2A and 2B, the vehicle seat 200 comprises a seat structure 202, a seat support member 204, and at least one mounting rod 210. The vehicle seat 200 further comprises an auscultation device 215, which comprises at least one microphone 208 and at least one tracking rod 210. It should be understood that the vehicle seat 200 is depicted in FIGS. 2A and 2B merely for illustration purposes, and the disclosed embodiments may suitably operate with a different configuration than that depicted in the figures.

The seat structure 202 represents the body of the vehicle seat 200. The seat structure 202 may include, for example, the seat cushion, springs, and other materials which determine the size and shape of the vehicle seat. The seat support member 204 represents the supporting skeleton of the vehicle seat 200. The seat support member 204 may be located inside the vehicle seat 200 and the seat structure 202, and may be coupled to the seat structure 202 through various direct or indirect connection points, for example. The seat support member 204 may include, for example, a series of rigid, or semi-rigid brackets, plates, rods, and any suitable equivalents thereof. In particular, the seat support member 204 may include at least one mounting rod 206. The mounting rods 206 extend in a longitudinal direction along the vehicle seat 200, and may be coupled to the seat support member 204 through various direct or indirect connection points, for example. The mounting rods 206 may include a suitable material for mounting components thereon, such as, for example, rigid or semi-rigid metal, plastic, or any equivalents thereof. In particular, the auscultation device 215 may be mounted to the mounting rods 206, as described further below.

The auscultation device 215 is configured to perform an auscultation examination procedure, i.e., a screening or examination, of a passenger in a vehicle seat by capturing a bodily sound of the passenger. As is understood to those of ordinary skill in the art, auscultation involves listening to the internal sounds of the passenger's body. Auscultation is typically performed for the purposes of examining the circulatory system (e.g., transportation of blood, nutrients, gases, and so forth, throughout the body), respiratory system (e.g., heart and lungs), and the gastrointestinal system (e.g., digestive system/bowels). Auscultation may be performed by an instrument that is configured to capture internal sounds of the body, such as a microphone. Ideally, the microphone, or other auscultation instrument, should be positioned according to the size and shape of the patient, as well as the organ(s) being examined. Therefore, an effective auscultation system should be adjustable according to specific examination parameters.

Moreover, as noted above, despite the importance of maintaining a healthy lifestyle, many people are simply too busy to receive regular medical check-ups. It is often the case that healthcare-related considerations are eschewed in view of one's daily obligations relating to, e.g., work, family, etc. Thus, there is currently a need to provide a manner in which people may receive regular, effective medical diagnosis check-ups, while minimizing interference with other non-health-related obligations.

Accordingly, in an effort to solve the above-referenced problems, the disclosed embodiments include an auscultation device 215 that is located inside the vehicle seat 200. The auscultation device 215 may perform an in-vehicle auscultation screening or examination as a part of a larger health care screening system, such as, for example, heartbeat monitoring, eye testing, blood alcohol testing, blood monitoring, and the like. The auscultation device 215 may include at least one microphone 208 and at least one tracking rod 210. The microphones 208 are movably coupled to the mounting rods 206, and may be movably coupled to the mounting rods 206 via the tracking rods 210. Specifically, the microphones 208 may be directly coupled to the tracking rods 210 via a suitable connection, such as, for example, a fastener, a bearing, a bracket, or any equivalents thereof. A single microphone 208 may be coupled to a single tracking rod 210, or a plurality of microphones may be coupled to a single tracking rod. The tracking rods 210 may be movably coupled to the mounting rods 206 via a suitable connection such as, for example, a fastener, a bearing, a bracket, or any equivalents thereof.

The microphones 208 include at least one microphone that is built directly into the vehicle seat 200. As such, the microphones 208 are able to perform an auscultation examination procedure upon a passenger by capturing a bodily sound, e.g., internal sounds of the body, of the passenger. In this way, the microphones 208 can be used in a manner similar to a stethoscope, as is commonly employed by medical professionals when performing auscultation-related procedures during a routine check-up, for example. Of course, the more microphones included in the microphone array 208, the more accurate the auscultation examination may become. The microphones 208 may be of any suitable type operable to capture a bodily sound of a passenger and transform the same into an electric signal for subsequent processing, e.g., transducer. The microphones 208 may also include a solenoid directly coupled to each microphone.

As referenced above, the microphones 208 and the tracking rods 210 may be movably coupled, either directly or indirectly, to the mounting rods 206 of the seat support member 204. The microphones 208 and the tracking rods 210 may be coupled to the mounting rods 206 in a movable manner such that the auscultation device 215 may be appropriately positioned according to the passenger, as well as the desired type of screening, e.g., the organ(s) being examined. An appropriate positioning of the microphones 208, in particular, may result in more accurate readings, and vice versa.

The position of the tracking rods 210 may be adjusted such that the tracking rods move in a substantially upward-and-downward direction, with respect to the mounting rods 206 (referring to a situation where the vehicle seat 200 is in use). For the purposes of the present disclosure, the substantially upward-and-downward direction may be referred to as a “first direction.” The tracking rods 210 may move along the mounting rods 206 in unison, i.e., each tracking rod 210 moves up or down in the same amount. Alternatively, the tracking rods 210 may move along the mounting rods 206 independently of one another, i.e., each tracking rod 210 may move in a different direction in a different amount. The freedom of movement permitted to the tracking rods 210 (and also the microphones 208) allows the auscultation device 215 to account for passengers of varied shapes and sizes.

Because the microphones 208 may be (movably) coupled to the tracking rods 210, the microphones 208 may move in the same substantially upward-and-downward direction with respect to the mounting rods 206 whenever the tracking rods 210 are adjusted. In other words, the microphones 208 and the tracking rods 210 move in unison with one another when the tracking rods are adjusted upwardly or downwardly. Therefore, both the microphones 208 and the tracking rods 210 are movable in the first direction.

Moreover, the microphones 208 are movably coupled to the tracking rods 210 such that the position of the microphones may be adjusted to move in a substantially left-and-right direction, with respect to the mounting rods 206 (referring to a situation where the vehicle seat 200 is in use). In other words, the microphones 208 are further configured to move independently of the tracking rods 210, and particularly, in a substantially left-and-right direction. For the purposes of the present disclosure, the substantially left-and-right direction may be referred to as a “second direction.” The microphones 208 may move along the tracking rods 210 in unison, i.e., each microphone 208 moves left or right in the same amount. Alternatively, the microphones 208 may move along the tracking rods 210 independently of one another, i.e., each microphone 208 may move in a different direction in a different amount. The freedom of movement permitted to the microphones 208 allows the auscultation device 215 to account for passengers of varied shapes and sizes. Therefore, the microphones 208 may be movably coupled to the mounting rods 206 via the tracking rods 210, such that the position of the microphones may move in the first direction (by virtue of their connection to the tracking rods), as well as the second direction (by virtue of their ability to “slide” along the tracking rods). In contrast, the tracking rods 210 are configured to move in the first direction along the mounting rods 206 only.

In this regard, FIGS. 2A and 2B depict two different configurations of the auscultation device 215 inside the same vehicle seat 200. FIG. 2A depicts a first auscultation device configuration 220 where the microphones 208 are spread apart from one another in the up-and-down direction (first direction) and the left-and-right direction (second direction). Because the microphones 208 are spread apart from one another in the up-and-down direction, it follows that the tracking rods 210 are also spread apart from one another in the up-and-down direction, as the two components are coupled to one another. The first auscultation device configuration 220 may be ideally suited for a larger driver with a wider and/or taller body.

On the other hand, FIG. 2B depicts a second auscultation device configuration 230 where the microphones 208 are close to one another in the up-and-down direction (first direction) and the left-and-right direction (second direction). Because the microphones 208 are close to one another in the up-and-down direction, it follows that the tracking rods 210 are also close to one another in the up-and-down direction, as the two components are coupled to one another. The second auscultation device configuration 230 may be ideally suited for a smaller driver with a thinner and/or shorter body.

The controller 100, as shown in FIG. 1, is configured to adjust the position of the auscultation device 215, so as to arrange the auscultation device according to the first or second auscultation device configuration 220/230, or any other suitable configuration. The controller 100 may adjust the position of the auscultation device 215 automatically and dynamically, based upon signaling discussed below in reaction to the size, shape, and health of the passenger. Further, the auscultation device 215 position may be determined based on a multitude of factors, such as a positioning of the passenger or a type of screening to be performed on the passenger, as described in further detail below.

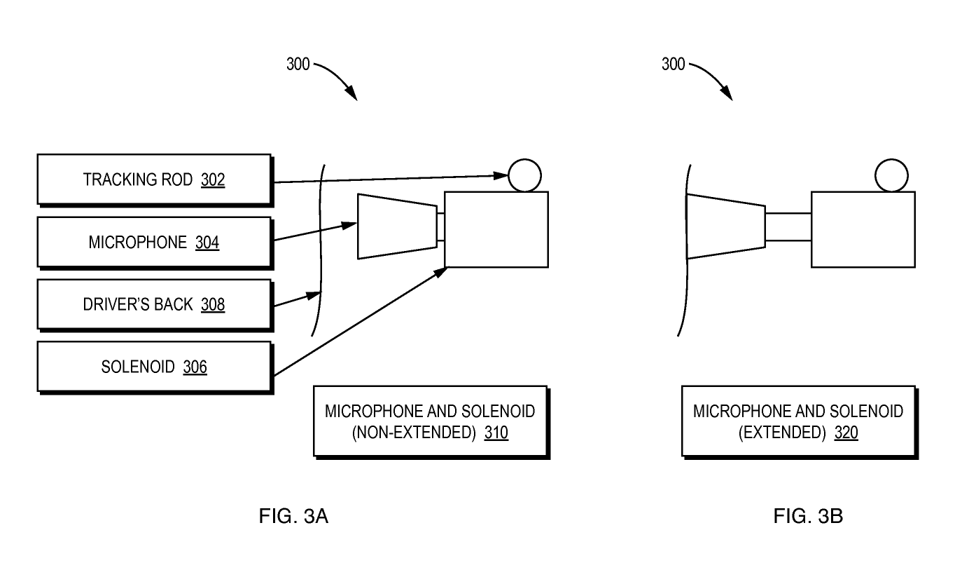

FIGS. 3A and 3B illustrate a side view of an exemplary simplified configuration of the auscultation device. As shown in FIGS. 3A and 3B, the auscultation device 300 includes a tracked rod 302 coupled to a microphone 304 and solenoid 306, positioned adjacent to a driver's back 308, shown for reference. The operation and configuration of the auscultation device 300 is equivalent to that of the auscultation device 215, as illustrated in FIGS. 2A and 2B. The auscultation device 300 is illustrated as merely a portion of an entire auscultation device disposed in a vehicle seat, and is depicted as such for illustration purposes only.

The microphone 304 is coupled to the tracking rod 304 in the same manner as illustrated in FIGS. 2A and 2B. The microphone 304 may include a solenoid 306 coupled directly thereto, and the body of the solenoid 306 may be connected to the tracking rod 202, accordingly. Alternatively, the auscultation device 300 may be configured such that the body of the microphone 304 is connected to the tracking rod 302. For the purposes of the present disclosure, the solenoid 306 may be considered a component of the microphone. Thus, operationally speaking, when it is stated that the microphone is movably coupled to the tracking rod, the tracking rod may be attached to the microphone, the solenoid, or any other component reasonably considered to be a segment thereof.

When viewed from the side, such that a front of the vehicle is on the left in FIGS. 3A and 3B, the microphone 304 may be positioned behind a passenger's back 308, such that the microphone can effectively capture a bodily sound of the passenger through the vehicle seat. The microphone 304 and solenoid 306 unit is extendable from the tracking rod 302 in a direction toward the passenger's back 308. As should be understood, an internal body sound may be more readily captured by the microphone 304 when it is extended from its default position, and thus positioned more closely to the passenger. As shown in FIG. 3A, the first auscultation device configuration 310 may illustrate the auscultation device 300 in an “inactive” state, whereby the microphone 304 and solenoid 306 are non-extended with respect to the passenger's back 308, and detracted toward the tracking rod 302. Conversely, as shown in FIG. 3B, the second auscultation device configuration 320 may illustrate the auscultation device 300 in an “active” state, whereby the microphone 304 and solenoid 306 are extended away from the tracking rod 302, and positioned more closely to the passenger's back 308.

Furthermore, the extended/non-extended positioning of the auscultation device 300 may be controlled by the controller 100. For example, when the auscultation device is inactive, e.g., not currently performing a screening of the passenger, the controller may set the position of the auscultation device 300 as in the first auscultation device configuration 310; whereas, when the auscultation device is active, e.g., currently performing a screening of the passenger, the controller may set the position of the auscultation device 300 as in the second auscultation device configuration 320. Notably, the tracking rod 302 is stationary in the front-and-back direction, when viewed from the side, as shown in FIGS. 3A and 3B. Thus, the microphone 304 may extend from the tracking rod 302, while the position of the tracking rod 302 remains on the same front-and-back plane.

Additionally, the angle of the microphone 304 and/or the solenoid 306 is adjustable in an upward-and-downward direction. As referenced above, the microphone 304 is movably coupled to the tracking rod 302. Thus, in addition to moving in a substantially up-and-down direction (first direction) and a substantially left-and-right direction (second direction), as well as extending toward/detracting away from the passenger, the angle of the microphone may also be adjusted so as to increase the effectiveness of a particular screening. For example, the microphone 304 may be set to a particular angle when screening a passenger's heart or lungs, while the microphone may be set to a different angle when screening the passenger's digestive system. Accordingly, the microphone 304 (and the solenoid 306) may be adjusted in the x-, y-, and z-planes, respectively. Of course, the adjustments of the positioning of the microphone 304, and also the entire auscultation device 300, may be effected by the controller 100.

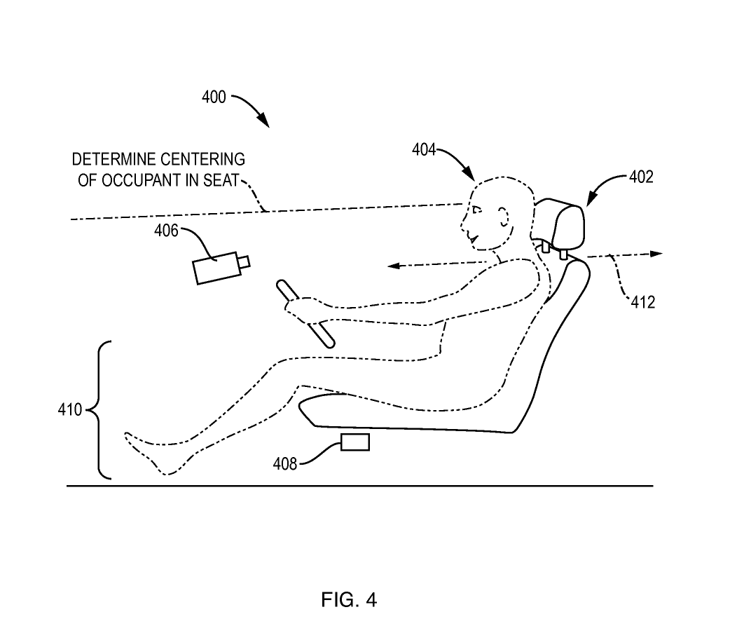

FIG. 4 illustrates an exemplary simplified configuration of a positioning assistance device. As shown in FIG. 4, vehicle arrangement 400 includes passenger 404 sitting in vehicle seat 402, the passenger being bisected by centerline 412, and positioning assistance device 410 including camera 406 and weight sensor 408. It should be understood that the vehicle arrangement 400 is depicted in FIG. 4 merely for illustration purposes, and the disclosed embodiments may suitably operate with a different configuration than that depicted in the figures.

The auscultation device, as shown in FIGS. 2A, 2B, 3A and 3B, is included in the vehicle seat 402, so as to perform a screening on the passenger 404. As described above, the controller 100 is configured to adjust a position of the auscultation device, whereby the positioning of the auscultation device may be based on various factors. One such factor is the positioning of the passenger. In this regard, the auscultation device, and particularly the microphones, need to be effectively positioned with respect to the passenger 404 to perform an accurate screening.

Accordingly, the positioning assistance device 410 is configured to detect a positioning of the passenger 404 while sitting in vehicle seat 402. The position assistance device 410 may detect the positioning of the passenger 404 by calculating a variety of positioning metrics, such as, for example, a centerline 412 of the passenger, i.e., a line which intersects a center point of the passenger in the left-and-right direction when facing the front of the passenger, a neck/shoulder line of the passengers, which may indicate a position of the heart and lungs, a height of the passenger, and the like. Upon detecting an indication, i.e., metric, of the positioning of the passenger 404, the positioning assistance device 410 may transmit the information which indicates the positioning to the controller 100. As shown in FIG. 1, the controller 100 is configured to receive such information from the positioning assistance device 410 via the vehicle communication interface 112. Upon receipt of the information which indicates the positioning of the passenger 404, the controller 100 may adjust the position of the auscultation device accordingly.

The positioning assistance device 410 may include at least one positioning detection instrument, such as, for example, the imaging device 406, e.g., camera, video camera, IR imager, scanner, etc., the weight sensor 408, and the like. Any suitable selection and configuration of position detection instruments may be utilized. The imaging device 406 may be positioned in front of and directed toward the passenger 404. Preferably, the imaging device 406 may be directed along an expected centerline 412 of the passenger 404. Program instructions may be stored in the controller 100 allowing the positioning assistance device 410 to calculate the centerline 412 of the passenger, the height of the passenger, etc., based on the images captured by the imaging device 406. The weight sensor 408 may be coupled to the vehicle seat 408. The weight sensor 408 may obtain information indicative of the size of the passenger 404. Similar to the imaging device 406, program instructions may be stored in the controller 100 allowing the positioning assistance device 410 to calculate the ideal left-and-right/up-and-down positioning of the microphones in the auscultation device, based on the data captured by the weight sensor 408.

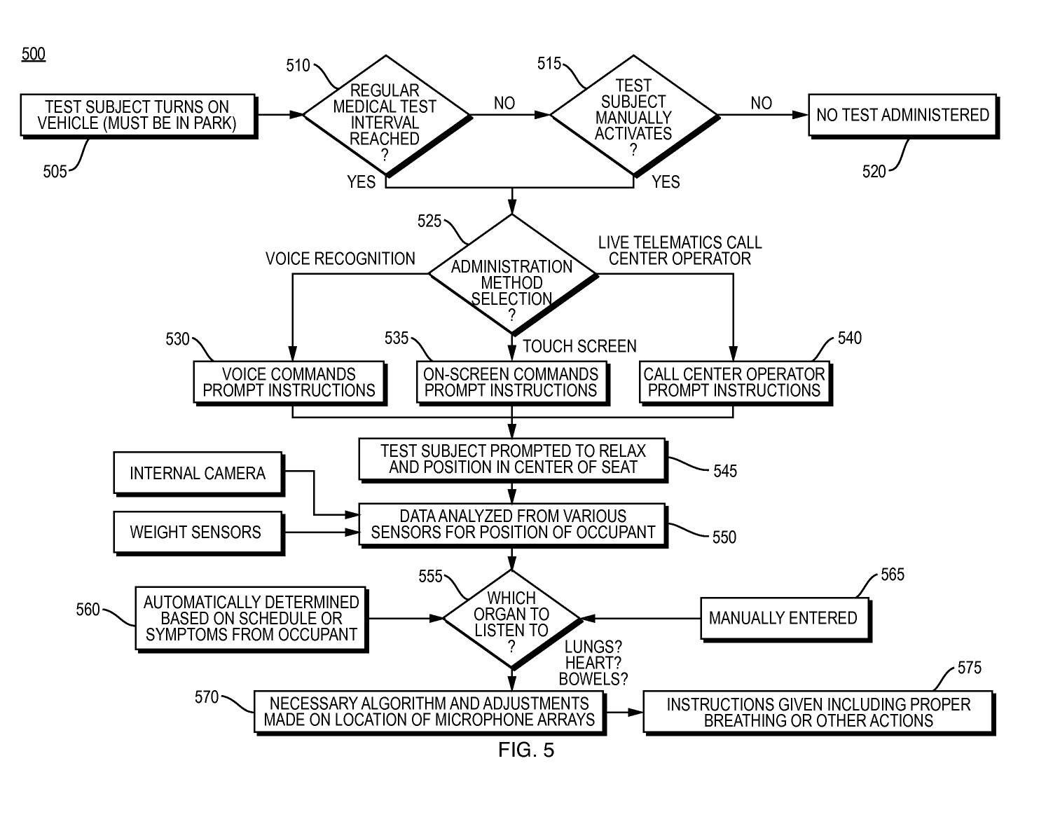

FIG. 5 illustrates an exemplary simplified procedure for calibrating the auscultation device and initiating a screening of a passenger. As shown in FIG. 5, the procedure 500 may start at Step 505, continue to Step 510, and so forth, where, as described in greater detail above, the auscultation device may be calibrated and a screening of a passenger may be initiated. Although FIG. 5 depicts steps in a particular order, it should be understood that the depicted embodiment is not limiting, and the particular order is depicted merely for illustration purposes.

At Step 505, the procedure 500 may commence when the test subject, e.g., passenger, turns on the vehicle. For safety purposes, in order for the procedure 500 to continue, the vehicle must be in park. At Step 510, it is determined whether a regular medical test interval, i.e., auscultation screening interval, has been reached. If the regular medical test interval has not yet been reached, at Step 515, it is determined whether the test subject manually activates an auscultation screening process. When the test subject has not manually activated an auscultation screening, at Step 520, it is determined that no screening is administered, and the procedure 500 ends.

Conversely, when the regular medical test interval has been reached, or the test subject has manually activated an auscultation screening, at Step 525, a method of administering/activating the auscultation screening may be selected. The controller 100, as shown in FIG. 1, may be configured to receive an initiation command. The initiation command may derive from the passenger or, alternatively, a remote source, such as a call center operator or a medical professional, or automated telematics server. At Steps 530, 535 and 540, an initiation command may be received via a voice command, an on-screen/touchscreen command, or a command from a remote call center operator/medical professional or automated telematics server, respectively. Upon receipt of the initiation command, the controller 100 is configured to initiate the auscultation screening of the passenger.

At Step 545, the test subject may be prompted, e.g., via a heads-up display, to relax and position himself/herself in the center of the vehicle seat. At Step 550, the passenger positioning detection process may begin. In particular, data may be captured by the positioning assistance device, as shown in FIG. 4, e.g., via an imaging device, a weight sensor, etc., and analyzed to detect the positioning of the test subject. As described above, various metrics may be calculated, such as, for example, a centerline, a height, and a neck/shoulder line of the passenger.

At Step 555, the type of screening to be performed is determined, or, more specifically, which organ(s) to be screened, e.g., heart, lungs, bowels, etc. The determination may be made automatically by telematics, as in Step 560, e.g., based on a predetermined schedule or on symptoms detected by the auscultation device. In the alternative, the determination may be manually entered by the passenger, as in Step 565. After determining the type of screening to be performed, Step 570 includes the controller adjusting a position of the auscultation device based on the determined type of screening. At Step 575, the passenger is instructed to relax, breathe properly, etc., as the auscultation screening begins.

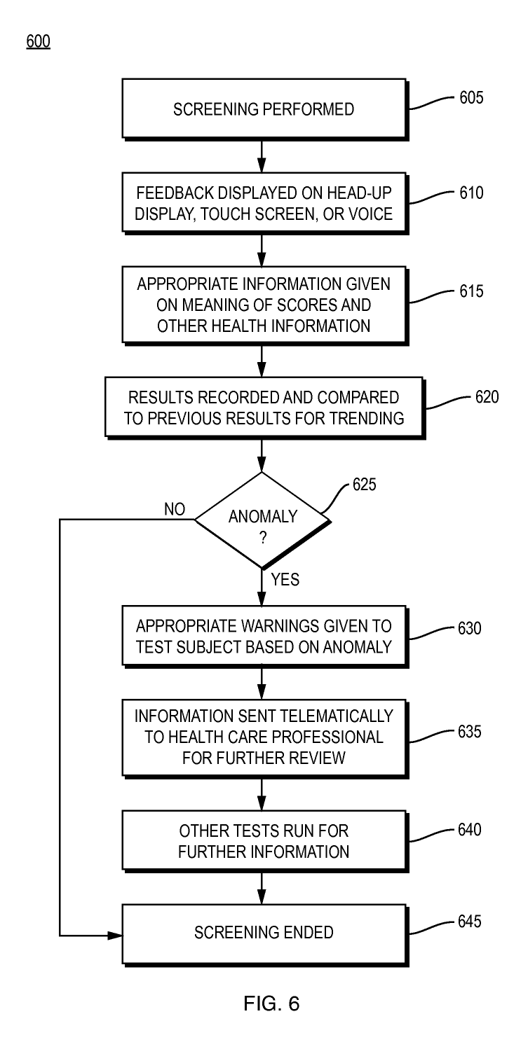

FIG. 6 illustrates an example simplified procedure for performing the screening of the passenger and determining a diagnosis of the passenger based on results of the screening. As shown in FIG. 6, the procedure 600 may start at Step 605, continue to Step 610, and so forth, where, as described in greater detail above, the screening of the passenger may be performed by the auscultation device and a diagnosis of the passenger may be determined based on results of the screening. Although FIG. 6 depicts steps in a particular order, it should be understood that the depicted embodiment is not limiting, and the particular order is depicted merely for illustration purposes.

At Step 605, the procedure 600 commences by performing an auscultation screening of a passenger in a vehicle seat by capturing a bodily sound of the passenger. The bodily sound of the passenger may be captured by at least one microphone, as described in detail above. Prior to initiating the screening, the auscultation device may be calibrated, e.g., adjusted, if necessary, as shown in FIG. 5.

After the screening is complete, at Step 610, feedback may be provided to the passenger based on results of the screening. Feedback may be communicated via any suitable means, such as, for example, a heads-up display, a touchscreen, audible feedback, etc. At Step 615, additional information relating to the feedback may be provided to the passenger, including contextual data to explain the meaning of the determined diagnosis.

At Step 620, the results of the screening, e.g., scores, warnings, etc., may be stored in a memory unit, such as the memory 140 coupled to the controller 100, a cloud-based server, the passenger's key fob, a server maintained by a health care professional, such as a telematics server, etc. The stored results may be compared to previously stored results for additional insight into the diagnosis by calculating health-related trends, for example.

At Step 625, the results of the screening are analyzed to determine whether an anomaly/irregularity in the health of the passenger may currently exist. If an irregularity exists, at Step 630, a warning notification is provided to the passenger via a suitable means, such as, for example, a heads-up display, a touchscreen, audible feedback, etc. Furthermore, at Step 635, the results of the screening may be transmitted to a health care professional for further review. The transmission may be performed utilizing, for example, the network interface 110 in the controller 100. The transmission may be performed via any suitable communications protocol, such as, for example, a wireless or wired protocol, Bluetooth, or other telematics-related protocols. This step may be useful for automatically notifying a health care professional in the case of an emergency, or for automatically updating a patient's medical records.

At Step 640, other tests may be initiated, e.g., additional screenings, upon detecting an irregularity in the passenger. An additional screening may be performed by the auscultation unit to obtain additional information relating to the same, or different, organ, as that which was tested. Or, the same screening may be performed a second time to confirm that the determined diagnosis based on the results of the screening is accurate. Moreover, an appointment for a future screening may be made. After this step, or if no irregularity is detected in the passenger, the screening is ended (Step 645).

It should be understood that one or more of the above steps may be performed by the controller 100, as shown in FIG. 1. It should also be understood that the steps shown in FIGS. 5 and 6 are merely examples for illustration, and certain steps may be included or excluded as desired. Further, while a particular order of the steps is shown, this ordering is merely illustrative, and any suitable arrangement of the steps may be utilized without departing from the scope of the embodiments herein. Moreover, while the procedures 500 and 600 are described separately, certain steps from each procedure may be incorporated into each other procedure, and the procedures are not meant to be mutually exclusive.

The techniques described herein, therefore, provide for performing auscultation using motor vehicle seats. As noted above, performing an auscultation screening on a passenger in a vehicle seat allows for an effective means of monitoring one's health, while reducing interference with the patient's potentially busy lifestyle. Moreover, a medical diagnosis based on results of the auscultation screening may be communicated to the passenger, e.g., through notifications or instructions; and, the diagnosis may be transmitted to a certified health care professional for review.

While there have been shown and described illustrative embodiments that provide for performing auscultation using motor vehicle seats, it is to be understood that various other adaptations and modifications may be made within the spirit and scope of the embodiments herein, with the attainment of some or all of their advantages. For instance, it is expressly contemplated that the components and/or elements described herein can be implemented as software being stored on a tangible, non-transitory computer-readable medium (e.g., disks/CDs/RAM/EEPROM/etc.) having program instructions executable by a controller, as described above, which may constitute hardware, firmware, or a combination thereof. Accordingly this description is to be taken only by way of example and not to otherwise limit the scope of the embodiments herein. Therefore, it is the object of the appended claims to cover all such variations and modifications as come within the true spirit and scope of the embodiments herein.

What is claimed is:

1. A system, comprising:
a vehicle seat including:
a seat support member that includes at least one mounting rod extending in a longitudinal direction; and
an auscultation device including at least one microphone that is movably coupled to the at least one mounting rod and configured to perform a screening of a passenger in the vehicle seat by capturing a bodily sound of the passenger; and
a controller configured to initiate the screening of the passenger and to diagnose the passenger based on results of the screening.

2. The system according to claim 1, wherein the auscultation device further comprises:
at least one tracking rod that is movably coupled to the at least one mounting rod and extends in a transverse direction with respect to the at least one mounting rod, wherein
the at least one microphone is movably coupled to the at least one mounting rod via the at least one tracking rod.

3. The system according to claim 2, wherein:
the controller is further configured to adjust the position of the auscultation device, wherein:
the at least one tracking rod is movable in a first direction with respect to the at least one mounting rod, and
the at least one microphone is movable in the first direction and a second direction with respect to the at least one mounting rod, the second direction being substantially orthogonal to the first direction.

4. The system according to claim 2, wherein:
a plurality of microphones are movably coupled to a single tracking rod.

5. The system according to claim 1, wherein:
the controller is further configured to provide feedback to the passenger based on the results of the screening.

6. The system according to claim 1, wherein:
the controller is further configured to transmit the results of the screening to a health care professional.

7. The system according to claim 1, wherein:
the controller is further configured to store the results of the screening in a memory unit.

8. The system according to claim 1, wherein:
the controller is further configured to compare the results of the screening to previously stored results.

9. The system according to claim 1, wherein:
the controller is further configured to initiate an additional screening of the passenger upon detecting irregularities in the passenger.

10. The system according to claim 1, wherein:
the controller is further configured to:
receive an initiation command which includes one of a voice command, an on-screen command, and an external command, and
initiate the screening of the passenger by the auscultation device upon receipt of the initiation command.

11. The system according to claim 1, wherein:
the controller is further configured to adjust a position of the auscultation device.

12. The system according to claim 11, wherein:
an angle of the at least one microphone is adjustable in an upward-and-downward direction.

13. The system according to claim 11, wherein:
the at least one microphone is extendable in a direction toward the passenger.

14. The system according to claim 1, further comprising:
a positioning assistance device that includes at least one of a camera directed toward the passenger and a weight sensor coupled to the vehicle seat, and is configured to detect a positioning of the passenger.

15. The system according to claim 14, wherein:
the positioning assistance device is further configured to transmit information indicating the positioning of the passenger, and
the controller is further configured to adjust the position of the auscultation device based on the positioning of the passenger.

16. The system according to claim 1, wherein:
the controller is further configured to determine a type of screening being performed on the passenger, wherein the determination is made automatically or based on input from the passenger.

17. The system according to claim 16, wherein:
the controller is further configured to adjust the position of the auscultation device based on the type of screening being performed on the passenger.

18. A method, comprising:
initiating, by a controller, a screening of a passenger in a vehicle seat by an auscultation device;
performing, by the auscultation device, the screening of the passenger by capturing a bodily sound of the passenger using at least one microphone; and
diagnosing, by the controller, the passenger based on results of the screening.

19. A non-transitory computer readable medium containing program instructions executable by a controller, the computer readable medium comprising:
program instructions that initiate a screening of a passenger in a vehicle seat by an auscultation device, the screening being performed by capturing a bodily sound of the passenger using at least one microphone; and
program instructions that diagnose the passenger based on results of the screening.
